(19) United States
(12) Patent Application Publication
(10) Pub. No.: US 2001/0014645 A1
(43) Pub. Date: Aug. 16, 2001

(54) Catalyst for converting aromatic hydrocarbon and conversion method thereof

(76) Inventors: Katuhiko Ishikawa, Yokohama-shi (JP); Eiji Yasui, Yokohama-shi (JP); Fumio Haga, Yokohama-shi (JP); Toshio Waku, Yokohama-shi (JP); Toshiyuki Enomoto, Yokohama-shi (JP)

Correspondence Address: OSTROLENK FABER GERB & SOFFEN, 1180 AVENUE OF THE AMERICAS, NEW YORK, NY, 100368403

(21) Appl. No.: 09/797,090

(22) Filed: Mar. 1, 2001

Related U.S. Application Data
(62) Division of application No. 09/452,484, filed on Dec. 1, 1999.

(30) Foreign Application Priority Data
Dec. 4, 1998 (JP) 10-346159

(51) Int. Cl. C07C 6/00

(52) U.S. Cl. 502/60; 585/420; 585/433

(57) ABSTRACT

There is provided a catalyst exhibiting a high activity and less catalytic deterioration, as well as a high selectivity, suitable for use in allowing aromatic hydrocarbons of 9 or more carbon atoms to react, and thereby converting them into toluene and aromatic hydrocarbons of 8 carbon atoms more useful as gasoline bases, and a conversion method using the catalyst. The catalyst is used for converting aromatic hydrocarbons of 9 or more carbon atoms in a material oil containing a component with a boiling point exceeding 210° C. into toluene and aromatic hydrocarbons of 8 carbon atoms in the presence of hydrogen, and contains a carrier containing one or more than one zeolites in which the maximum pore diameter of micropores is in a range of 0.6 to 1.0 nm; and one or more than one metals selected from the Group VIA metals of the Periodic Table or compounds thereof.

### BACKGROUND OF THE INVENTION

1. Field of the Invention

The present invention relates to a catalyst for converting aromatichydrocarbons, and a conversion method using the catalyst. More particularly, it relates to a catalyst suitable for use in converting aromatic hydrocarbons of 9 or more carbon atoms into toluene and aromatic hydrocarbons of 8 carbon atoms more useful as gasoline bases, and a method of converting aromatic hydrocarbons using the catalyst.

2. Background Art

Aromatic hydrocarbons contained in gasoline bases generally have high octane values, and are superior as gasoline bases because of their high calorific values. Among them, toluene and aromatic hydrocarbons of 8 carbon atoms especially have higher octane values, and drive-ability levels, and thus their contents are desirable to be increased. In this regard, methods of directly converting aromatic hydrocarbons of 9 or more carbon atoms in gasoline fractions into toluene and aromatic hydrocarbons of 8 carbon atoms are of great significance.

On the other hand, in line with a move to regulate the benzene content of gasoline to 1% or less for environmental protection, some petroleum companies have already conducted the extraction of benzene from gasoline bases. Although benzene is used as a chemical raw material, there is the fear of its price reduction due to the oversupply thereof in the future. For this reason, a method of directly converting benzene and aromatic hydrocarbons of 9 or more carbon atoms in gasoline fractions into toluene and aromatic hydrocarbons of 8 carbon atoms is especially of great significance.

The process in which aromatic hydrocarbons are allowed to react with each other to be converted into aromatic hydrocarbons having different numbers of carbon atoms from the reactant aromatic hydrocarbons is accomplished by the transalkylation reaction and disproportionation reaction of aromatic hydrocarbons. The transalkylation reaction allows a plurality of different aromatic hydrocarbons to react with each other, while the disproportionation reaction of aromatic hydrocarbons is effected between two molecules of the same aromatic hydrocarbons. A well known process regarding these reactions is the manufacture of benzene and xylene utilizing the disproportionation reaction of toluene. Further, as an application of this reaction, there also exists a method of increasing the yield of xylene through inducing the transalkylation reaction by adding aromatic hydrocarbons of 9 or more carbon atoms to the starting material.

However, this process does not require benzene as a starting material, but requires toluene which itself is useful as a gasoline base. It also employs highly purified materials because its object is to synthesize chemical industrial starting materials. Accordingly, little attention has been paid as to using gasoline fractions, etc., which are mixtures of other compounds, especially various hydrocarbons including aliphatic hydrocarbons as starting materials.

In Japanese Patent Application Laid-Open No. Sho 60-246330, there is disclosed a method of manufacturing benzene and methyl-substituted benzene characterized in that heavy aromatic hydrocarbons of 9 or more carbon atoms are hydrotreated in the presence of a crystalline aluminosilicate catalyst carrying at least one metal selected from the Group VIII metals of the Periodic Table. In this publication, however, there is no description on the pore diameter of aluminosilicate, and there is revealed no findings on the effects of the pore diameter. There is also a deficiency in that the conversion ratio of trimethylbenzene is low.

The U.S. Pat. No. 5,347,061 discloses a method of directly converting benzene and aromatic hydrocarbons of 9 or more carbon atoms in gasoline fractions into toluene and aromatic hydrocarbons of 8 carbon atoms. The disclosed method relates to a process of manufacturing toluene and aromatic hydrocarbons of 8 carbon atoms by effecting transalkylation reaction, alkylation reaction, and decomposition reaction of a benzene rich fraction of hydrocarbons of 6 carbon atoms obtained by distilling reformed gasoline and a fraction of mixtures of aromatic hydrocarbons of 9 or more carbon atoms and non-aromatic hydrocarbons in the presence of an acidic metarosilicate catalyst. As for the acidic metarosilicate catalysts, there are disclosed zeolite catalysts such as ZSM-5, but no detailed description is given to the pore diameter or carrying of a metal.

In Japanese Patent Application Laid-Open No. Hei 9-155198, there is disclosed a catalyst which comprises a carrier containing at least one zeolite in which the maximum pore diameter of micropores is in a range of 0.6 to 1.0 nm, and a SiO₂/Al₂O₃ ratio is 50 or more, and at least one metal selected from the Groups VIII and VIA metals of the Periodic Table or a compound thereof, the metal or compound being carried on the carrier. This catalyst is an aromatic hydrocarbon converting catalyst for use in a method of converting aromatic hydrocarbons of 9 or more carbon atoms contained in a raw material oil having a boiling point ina range of 100 to 210° C. and containing no benzene into toluene and aromatic hydrocarbons of 8 carbon atoms in the presence of hydrogen.

In Japanese Patent Application Laid-Open No. Hei 9-38505, there is also disclosed a catalyst which comprises a carrier containing at least one zeolite in which the maximum pore diameter of micropores is in a range of 0.6 to 1.0 nm, and a SiO₂/Al₂O₃ ratio is 50 or more, and at least one metal selected from the Groups VIII and VIA metals of the Periodic Table or a compound thereof, the metal or compound being carried on the carrier. This catalyst is an aromatic hydrocarbon converting catalyst for use in a method of converting benzene and aromatic hydrocarbons of 9 or more carbon atoms contained in a raw material oil having a boiling point in a range of 30 to 210° C. into toluene and aromatic hydrocarbons of 8 carbon atoms in the presence of hydrogen.

The aforementioned catalysts disclosed in Japanese Patent Application Laid-Open Nos. Hei 9-155198 and 9-38505 provides a high conversion ratio of aromatic hydrocarbons of 9 or more carbon atoms, especially trimethylbenzene. However, the disclosed catalysts have been required to be further improved in that they undergo significant catalytic deterioration when used for raw material oils containing heavier fractions with an upper limit of the boiling point range exceeding 210° C.

### SUMMARY OF THE INVENTION

It is a first object of the present invention to provide a catalyst exhibiting a high activity and less catalytic deterioration, and having a high selectivity, suitable for use in allowing aromatic hydrocarbons of 9 or more carbon atoms to react, thereby converting them into toluene and aromatic hydrocarbons of 8 carbon atoms more useful as gasoline bases.

It is a second object of the present invention to provide a method of allowing aromatic hydrocarbons of 9 or more carbon atoms to react using the catalyst, thereby converting them into toluene and aromatic hydrocarbons of 8 carbon atoms more useful as gasoline bases.

The present inventors have conducted a close study on a novel catalyst for use in allowing aromatic hydrocarbons of 9 or more carbon atoms to react, thereby directly converting them into toluene and aromatic hydrocarbons of 8 carbon atoms more useful as gasoline bases. As a result, they have found that a catalyst comprising a carrier containing zeolite with a large maximum pore diameter of micropores and a large SiO₂/Al₂O₃ ratio, and a specific metal exhibits a high activity and less catalytic deterioration and has a high selectivity when used in the reaction of converting aromatic hydrocarbons of 9 or more carbon atoms in a raw material oil having an upper limit of boiling point range exceeding 210° C. into toluene and aromatic hydrocarbons of 8 carbon atoms. Further, they have also found that the catalyst especially exhibits a high activity and less catalytic deterioration, and has a high selectivity when used in the reaction of converting aromatic hydrocarbons of 9 or more carbon atoms and benzene in the raw material oil having an upper limit of boiling point range exceeding 210° C. into toluene and aromatic hydrocarbons of 8 carbon atoms. Thus, the present invention has been achieved.

According to a first aspect of the present invention, there is provided a catalyst for converting aromatic hydrocarbons of 9 or more carbon atoms in a material oil containing a component with a boiling point exceeding 210° C. into toluene and aromatic hydrocarbons of 8 carbon atoms in the presence of hydrogen, wherein the catalyst comprises: a carrier containing one or more than one zeolites in which the maximum pore diameter of micropores is in a range of 0.6 to 1.0 nm; and one or more than one metals selected from the Group VIA metals of the Periodic Table or compounds thereof.

According to a second aspect of the present invention, there is provided a method of converting aromatic hydrocarbons of 9 or more carbon atoms in a material oil containing a component with a boiling point exceeding 210° C. into toluene and aromatic hydrocarbons of 8 carbon atoms in the presence of hydrogen using a catalyst, wherein the catalyst comprises: a carrier containing one or more than one zeolites in which the maximum pore diameter of micropores is in a range of 0.6 to 1.0 nm; and one or more than one metals selected from the Group VIA metals of the Periodic Table or compounds thereof.

According to a third aspect of the present invention, there is provided the method of converting aromatic hydrocarbons according to the foregoing second aspect of the invention, wherein aromatic hydrocarbons of 9 or more carbon atoms and benzene in the material oil containing a component with a boiling point exceeding 210° C. are converted into toluene and aromatic hydrocarbons of 8 carbon atoms in the presence of hydrogen.

By using the catalyst for converting aromatic hydrocarbons according to the present invention, it is possible to convert aromatic hydrocarbons of 9 or more carbon atoms in a raw material oil containing a component with a boiling point exceeding 210° C., into toluene and aromatic hydrocarbons of 8 carbon atoms more useful as gasoline bases through transalkylation reaction and dealkylation reaction in the presence of hydrogen with high efficiency. Moreover, the catalyst undergoes less deterioration and has a longer life.

Further, the catalyst for converting aromatic hydrocarbons according to the present invention can be preferably used in achieving highly efficient conversion into toluene and aromatic hydrocarbons of 8 carbon atoms through transalkylation reaction and dealkylation reaction in the presence of hydrogen using a raw material oil containing aromatic hydrocarbons of 9 or more carbon atoms and benzene.

### BRIEF DESCRIPTION OF THE DRAWINGS

These and other objects and advantages of the present invention will become clear from the following description with reference to the accompanying drawings, wherein:

FIG. 1 is a conceptual diagram for illustrating a manufacturing method of a gasoline base using a catalyst of the present invention; and

FIG. 2 is a conceptual diagram for illustrating another manufacturing method of a gasoline base using the catalyst of the present invention.

### DETAILED DESCRIPTION OF THE PREFERRED EMBODIMENTS

Hereinbelow, the present invention will be described in details.

A catalyst according to the present invention is characterized by including a carrier containing one or more than one zeolites with a maximum pore diameter of micropores in a range of 0.6 to 1.0 nm, and one or more than one metals selected from the Group VIA metals of the Periodic Table or compounds thereof.

The porous structures of the materials for porous catalysts are generally classified into flow route, macro-pore (porous diameter exceeding 50 nm), meso-pore (porous diameter ranging from 2 to 50 nm), and micropore (porous diameter less than 2 nm). Some porous catalyst materials have two or more different-sized micropores, and in the present invention, the maximum pore diameter of micropores denotes the porous entrance diameter of the largest one of these micropores. In the present invention, when the micropore is not a perfect circle but an ellipse, a major axis there of is referred to as a porous diameter. In the micropore of zeolite used in the present invention, the ring forming the porous entrance is generally comprised of 12-membered ring of oxygen atoms.

The maximum porous diameter of micropores of zeolite to be used in the present invention needs to be in a range of 0.6 to 1.0 nm, preferably in a range of 0.6 to 0.8 nm. When it is less than 0.6 nm, undesirably, the reaction activity in the conversion from aromatic hydrocarbons of 9 or more carbon atoms into toluene and aromatic hydrocarbons of 8 carbon atoms is decreased and the selectivity of decomposition reactions of aliphatic hydrocarbons co-present in the starting material becomes higher.

Examples of zeolite to be used in the present invention include mordenite, X-type zeolite, Y-type zeolite, offretite, β-typezeolite, L-typezeolite, and Ω-type zeolite. Among them, mordenite, Y-type zeolite, and β-type zeolite are preferred, and mordenite is more preferred. These zeolites may be either synthesized or natural products. In addition, in the present invention, these zeolites may be used singly, or if required, in combinations of two or more thereof.

The SiO₂/Al₂O₃ ratio (on a mole basis) in zeolite used in the present invention is preferred to be larger in terms of the catalytic activity, catalytic deterioration rate, and stability during catalyst regeneration. The SiO₂/Al₂O₃ratio is preferred to be 10 or more, more preferred 15 or more, particularly preferred 50 or more, more particularly preferred 120 to 300, and most preferred 150 to 250. Zeolites having such a large SiO₂/Al₂O₃ ratio may be synthesized, if possible, by appropriately adjusting the ratio of the starting materials during synthesis so as to obtain a desired value, but may also be obtained by synthesizing zeolites having a smaller SiO₂/Al₂O₃ ratio and subjecting them to dealuminization by known methods such as steaming or acid treatment. In the present invention, the dealuminization treatment is preferably conducted.

In general, zeolite has a negative charge in the crystalline framework wherein there exists a counter cation to compensate it. Although the counter cation in zeolite used in the present invention can be arbitrarily selected, it is preferably a hydrogen ion, or a mixture of a hydrogen ion and an alkaline metal ion and/or alkaline earth metal ion, and more preferably a mixture of a hydrogen ion and an alkaline metal ion and/or alkaline earth metal ion.

The ratio of hydrogen ions to total cations is preferably 20% or more, more preferably in a range of 30 to 99.5%, and most preferably in a range of 40 to 99% on an ion equivalent basis.

The ratio of alkaline metal ions and/or alkaline earth metal ions to total cations is preferably 80% or less, more preferably in a range of 0.5 to 70%, and most preferably in a range of 1 to 60% on an ion equivalent basis. As counter cations, K⁺ and Na⁺ are preferred among alkaline metal ions, while Mg²⁺ and Ca²⁺ are preferred among alkaline earth metal ions.

A zeolite containing the foregoing alkaline metal ions and/or alkaline earth metal ions as counter cations can be prepared by exchanging a part of hydrogen ions with alkaline metal ions and/or alkaline earth metal ions when the counter cations in the material zeolite are mostly hydrogen ions.

On the other hand, when the counter cations in the material zeolites are mostly alkaline metal ions and/or alkaline earth metal ions, these ions may be exchanged with hydrogen ions so as to be partially left. Alternatively, once almost all of these ions are exchanged with hydrogen ions, and then exchanged with alkaline metal ions and/or alkaline earth metal ions again.

The exchange of counter cations with hydrogen ions can be accomplished by another known method where the counter cations are exchanged with ammonium ions, and then converted into hydrogen ions by calcination, in addition to the method of directly exchanging into hydrogen ions.

The carrier of the catalyst according to the present invention can be molded using commonly used molding methods. Examples of the molding methods include extrusion molding, tabletting, and oil dropping. Among them, extrusion molding is preferred. Binders may also be used in molding, if required.

There is no particular restriction as to the binders, and any kinds of binders including metal oxides such as alumina, titania, and silica-alumina, and clay minerals can be used. Among them, alumina, silica, and clay minerals are preferred, and alumina is particularly preferred. Further, two or more binders may also be used, if required. Although there is no restriction as to the amount of the binder to be used, it is preferably in a range of 5 to 50% by mass, and more preferably in a range of 10 to 30% by mass based on the total mass of the entire carrier including the binder. The catalytic performance may be reduced when the amount of the binder used is too large, or the molding may become difficult when it is too small.

The catalyst according to the present invention comprises a carrier comprised of the zeolite molded body, and one or more than one metals selected from the Group VIA metals of the Periodic Table or compounds thereof. It is undesirable that the catalyst contains no metal, because no metal content thereof reduces the catalytic activity and makes the catalyst to be more likely to deteriorate.

Although the metal may be carried on the carrier, or may also be physically mixed with the carrier, it is preferably carried on the carrier. When the metal is physically mixed therewith, it is preferably carried on another carrier comprised of a stable metal oxide and the like.

There is no particular restriction as to the method of carrying metals, and conventional carrying methods can be employed. Examples thereof include impregnation method and CVD (Chemical Vapor Deposition) method. Out of these methods, the impregnation method is preferred. Preferable examples of the impregnation method include adsorption method, ion exchange method, vaporizing-drying method, incipient wetness method, and spray method.

The metals to be used for the catalyst according to the present invention is one or more than one metals selected from the Group VIA metals of the Periodic Table. Group VIA metals of the Periodic Table are Cr, Mo, and W. Among them, Mo and W are preferred, and Mo is more preferred.

These metals can be used singly, or in combinations with two or more thereof. Alternatively, they can be used in combination with other metals, if required. However, it is not preferable that other metals are used in combination.

Although starting materials for the metals to be used may be any commonly used ones, water-soluble compounds are preferred from the viewpoint of catalyst preparation. Preferable examples of starting materials for the metals include chlorides, nitrates, sulfates, oxides, carbonyl compounds, and ammine complexes. In addition, preferable examples of the starting materials for the metals further include oxygen acids such as chromic acid, molybdic acid, and tungstic acid, and salts thereof. More preferable examples thereof include ammonium salts of oxygen acids of the group VIA metals.

In the catalyst according to the present invention, although the metals may be present in the form of metal atoms, or in the form of various compounds thereof, they are preferably present in the form of metal atoms, oxides, and sulfides among them in terms of the reaction activity.

The content of the group VIA metals and compounds thereof is appropriately selected depending upon the kinds of metals, reaction conditions, and the like, and has no restriction. However, it is preferably in a range of 0.3 to 10% by mass, more preferably in a range of 0.5 to 8% by mass, and most preferably in a range of 1 to 5% by mass in terms of metals for respective metal species based on the total mass of the catalyst.

The catalyst to be used in the present invention is preferably subjected to a calcination treatment after being prepared. The calcination temperature is generally in a range of 100 to 600° C., and preferably in a range of 200 to 550° C. It is also preferably hydrotreated or subjected to a sulfurization treatment as a pretreatment prior to the start of a reaction.

The hydrotreating is conducted generally at a treating temperature of 100 to 600° C., and preferably 200 to 500° C. The sulfurization treatment is preferably conducted using sulfurization agents such as hydrogen sulfide, carbon disulfide, and dimethyl disulfide (DMDS) in the presence of a gas containing a hydrogen gas. The sulfurization temperature is preferably in a range of 100 to 500° C., and more preferably 200 to 400° C.

The catalyst according to the present invention is preferably used in a method of converting aromatic hydrocarbons of 9 or more carbon atoms in a material oil into toluene and aromatic hydrocarbons of 8 carbon atoms, especially in the presence of benzene.

In the present invention, the material oil contains a component having a boiling point exceeding 210° C. The boiling point herein referred to is determined according to the measurement method specified by ASTM D86. The boiling point of the raw material oil may fall in any range as long as it satisfies this condition. However, the boiling point of the material oil usable is preferred to be in a range of 30 to 260° C., more preferred to be 40 to 250° C., and particularly preferred to be 50 to 240° C. in values defined as an initial boiling point and an end point in the measurement method specified in ASTM D86.

The catalyst according to the present invention is characterized by having a long catalytic life even when the catalyst is used for conversion of a material oil containing a fraction with a high boiling point. However, too high content of the fraction with a high boiling point is undesirable because it shortens the catalytic life.

The material oil to be used in the present invention is required to contain aromatic hydrocarbons of 9 or more carbon atoms. Although the content of aromatic hydrocarbons of 9 or more carbon atoms in the material oil can be arbitrarily adopted, in general, the more, the better in terms of the reaction efficiency. In the present invention, the content of aromatic hydrocarbons of 9 or more carbon atoms is generally 1% by mass or more, preferably 30% by mass or more, and more preferably 50% by mass or more based on the total mass of the material oil (including recycled produced oil when recycling of the produced oil is conducted). Especially, it is desirable that the content of aromatic hydrocarbons of 9 carbon atoms is 1% by mass or more, preferably 30% by mass or more, and more preferably 50% by mass or more based on the total mass of the material oil.

Among the aromatic hydrocarbons of 9 or more carbon atoms in the material oil, aromatic hydrocarbons of 9 to 11 carbon atoms are preferred, and aromatic hydrocarbons of 9 to 10 carbon atoms are more preferred. Further, aromatic hydrocarbons having methyl groups are preferred, and aromatic hydrocarbons rich in methyl groups are more preferred. Preferable examples thereof specifically include trimethylbenzene, methylethyl benzene, tetramethylbenzene, and dimethylethyl benzene.

It is not a necessary condition but preferable for the material oil used in the present invention to contain benzene in terms of the reaction efficiency. Although the benzene content of the material oil is arbitrarily selected, in general, the more, the better in terms of the reaction efficiency. In the present invention, the benzene content is generally 1% by mass or more, preferably 5% by mass or more, and more preferably 10% by mass or more based on the total mass of the material oil.

The ratio of aromatic hydrocarbons of 9 or more carbon atoms to benzene in the material oil (the number of moles of aromatic hydrocarbons of 9 or more carbon atoms/the number of moles of benzene) is preferably in a range of 0.5 to 10, and more preferably in a range of 1 to 5.

The aforementioned material oil to be used in the present invention may be a pure product such as a chemical industrial starting material. However, the material oil to be preferably used in the present invention is a mixture with other compounds. Examples thereof include petroleum distillates obtained by distilling crude oil, and oils obtained by subjecting petroleum distillates to various treatments. Among them, reformed gasoline obtained from a catalytic reforming apparatus, catalytic cracked gasoline obtained from a fluid catalytic cracking apparatus, and the like are preferably used, and in particular, reformed gasoline rich in aromatic components is preferably used.

In general, the aforementioned mixture also contains toluene and aromatic hydrocarbons of 8 carbon atoms, i.e., reaction products in accordance with the present invention. In the present invention, the mixture containing these compounds may be used as the material oil without further purification, but it is preferably used as the material oil after removal of these compounds therefrom by distillation or the like in terms of the reaction efficiency.

More specifically, reformed gasoline or the like is distilled to be separated into a fraction containing benzene, a fraction containing toluene and aromatic hydrocarbons of 8 carbon atoms, a fraction containing aromatic hydrocarbons of 9 or more carbon atoms, and the like. Among them, the fraction containing aromatic hydrocarbons of 9 or more carbon atoms is preferably used as the material oil.

Alternatively, the fraction containing aromatic hydrocarbons of 9 or more carbon atoms is more preferably mixed with the fraction containing benzene to be used as the material oil. Further, if required, high purity benzene separated from the benzene-containing fraction by known methods such as sulfolane extraction may also be mixed with the fraction containing aromatic hydrocarbons of 9 or more carbon atoms to be used as the material oil. It is noted that, toluene and aromatic hydrocarbons of 8 carbon atoms herein removed may be used as gasoline bases, individually, or may also be used as the chemical industrial starting materials.

The aromatic hydrocarbon conversion reaction using the catalyst according to the present invention is required to be carried out in the presence of a hydrogen gas. Although the ratio of the hydrogen gas to the material oil (volume of hydrogen/volume of the material oil at standard conditions of 0° C. and 1 atm) has no particular restriction, it is preferably in a range of 50 to 2000 Nm³ /m³, and more preferably in a range of 300 to 1500 Nm³ /m³. When the ratio of hydrogen gas to the material oil is too small, the catalytic life becomes extremely shorter. On the other hand, too large ratio thereof is not economical.

The catalyst according to the present invention should be used in the presence of hydrogen, and other reaction conditions can be appropriately selected depending upon the catalytic activity, compositions of the material oil and produced oil, and the like. There is no particular restriction as to reaction pressure, reaction temperature, liquid hourly space velocity (LHSV), and the like. However, the reaction pressure is generally in a range of 0.1 to 6 MPa, and preferably in a range of 0.5 to 4 MPa.

The reaction temperature is generally in a range of 200 to 550° C., preferably in a range of 250 to 500° C., and more preferably in a range of 350 to 490° C.

The LHSV is preferably in a range of 0.1 to 10 h⁻¹, and more preferably in a range of 0.5 to 5 h⁻¹.

Although the reactors used in the present invention can be any one of the fixed bed, fluidized bed, or expansion bed type, but preferably of the fixed bed type. The contact of the material oil with hydrogen and the catalyst may be accomplished by any methods of parallel elevation flow, parallel descending flow, and countercurrent flow. The reaction may be carried out by either a flow method or batch method, but the flow method is preferred.

When aromatic hydrocarbons of 9 or more carbon atoms is converted into toluene and aromatic hydrocarbon of 8 carbon atoms using the catalyst according to the present invention, the conversion ratio is higher with smaller content of unreacted aromatic hydrocarbons of 9 or more carbon atoms in the produced oil. Thus, although the produced oil is not necessarily required to be recycled and mixed with the raw material oil for another reaction, it is preferably recycled in order to raise the reaction efficiency.

When recycling is conducted, a part of the produced oil maybe directly mixed with the material oil, or only the fraction containing unreacted aromatic hydrocarbons of 9 or more carbon atoms is separated by distilling the produced oil, or the like, to be mixed with the material oil. Further, the whole fractions containing unreacted aromatic hydrocarbons of 9 or more carbon atoms may be mixed with the material oil, or only a part thereof may be mixed with the material oil.

It is preferred not to recycle a benzene-containing fraction when the benzene concentration in the produced oil is low, because the low benzene content thereof accelerates the coexisting aliphatic hydrocarbons of 6 or 7 carbon atoms (to serve as a gasoline base) to decompose into gaseous matters of inferior value, resulting in a reduced yield of gasoline bases. On the other hand, when high purity benzene separated from the benzene-containing fraction by known methods such as sulfolane extraction is used as a starting material, it is preferred to conduct recycling in terms of the reaction efficiency because the foregoing problem does not much matter in this case.

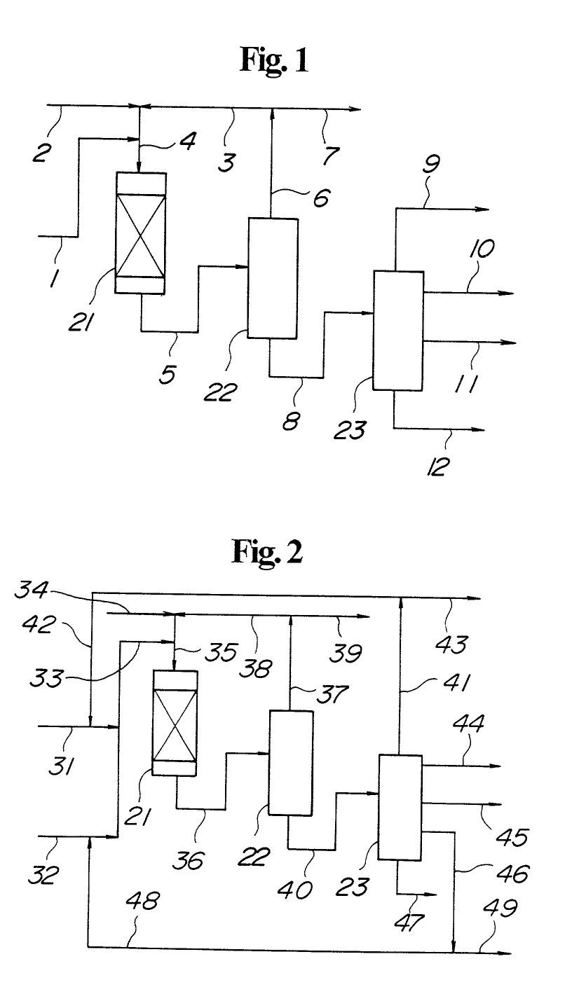

Referring now to FIG. 1, there is shown one embodiment of a simplified conceptual flow diagram illustrating a process in which a product is not recycled using the catalyst according to the present invention.

A material oil containing aromatic hydrocarbons of 9 or more carbon atoms (which may also include benzene) (in a conduit 1) is supplied via a conduit (4) to a reactor (21) along with make-up hydrogen (in a conduit 2) and a hydrogen rich gas (in a conduit 3) recycled from a separator (22). The produced oil is supplied via a conduit (5) to the separator (22), where the hydrogen rich gas (in a conduit 6) is separated therefrom, and the remaining produced oil is supplied via a conduit (8) to a distilling tower (23). The hydrogen rich gas is recycled via the conduit (3), while partially being bled (via a conduit 7). At the distilling tower (23), the produced oil is separated into a fraction containing benzene (into a conduit 9), a fraction containing toluene (into a conduit 10), a fraction containing aromatic hydrocarbons of 8 carbon atoms (into a conduit 11), and a fraction containing aromatic hydrocarbons of 9 or more carbon atoms (into a conduit 12).

FIG. 2 shows another embodiment of a simplified flow diagram illustrating a process in which a product is recycled using the catalyst according to the present invention.

Benzene (in a conduit 31) combines with a fraction containing benzene recycled via a conduit (42) from the distilling tower (23), while the material oil containing aromatic hydrocarbons of 9 or more carbon atoms (in a conduit 32) combines with a fraction containing aromatic hydrocarbons of 9 or more carbon atoms recycled via a conduit (48) from the distilling tower (23). These further combine with each other (via a conduit 33) to be transferred via a conduit (35) along with make-up hydrogen (in a conduit 34) and a hydrogen rich gas recycled via a conduit (38) from the separator (22) to the reactor (21).

The produced oil is supplied via a conduit (36) to the separator (22), where a hydrogen rich gas is separated via a conduit (37) therefrom, and the remaining produced oil is supplied via a conduit (40) to the distilling tower (23). The hydrogen rich gas is recycled via the conduit (38), while partially being bled (via a conduit 39). At the distilling tower (23), the produced oil is separated into a fraction containing benzene (into a conduit 41), a fraction containing toluene (into a conduit 44), a fraction containing aromatic hydrocarbons of 8 carbon atoms (into a conduit 45), a fraction containing aromatic hydrocarbons of 9 or more carbon atoms (into a conduit 46), and a heavy fraction (into a conduit 47). The fraction containing benzene (in the conduit 41) is recycled via the conduit (42), while partially being bled (via a conduit 43), if required. The fraction containing aromatic hydrocarbons of 9 or more carbon atoms (in the conduit 46) is recycled via a conduit (48), while partially being bled via a conduit (49), if required.

### EXAMPLES

Below, the present invention will now be described more specifically by way of examples and comparative examples, which should not be construed as limiting the scope of the invention.

### Example 1

To hydrogen-ion-type mordenite (HSZ690HOA, obtained from Toso K.K.) in which the maximum pore diameter of its micropores was 0.70 nm and the SiO₂/Al₂O₃ ratio was 203, boehmite deflocculated with dilute nitric acid was added, and the mixture was kneaded, extrusion molded, dried and calcinated to prepare a catalyst carrier. The amount of boehmite was adjusted so that the amount of alumina binder could be 30% by mass based on the total mass of the carrier. The resulting carrier is allowed to carry 3% bymass of Mo (basedon the total mass of the catalyst) in terms of metals using an aqueous solution of (NH₄)₆Mo₇O₂₄ by the Incipient Wetness method, dried and calcinated to prepare a catalyst. Then, prior to the following reaction, the catalyst was treated with a hydrogen gas at 220° C. for 1 hour.

The material oil (containing a component with an end point (EP) of 232.5° C. and a boiling point exceeding 210° C.) with a composition shown in FIG. 1 was used to conduct a reaction experiment using a 20-ml pressurized distribution type reaction apparatus. The material oil is prepared by mixing benzene with a fraction mainly containing aromatic hydrocarbons of 9 carbon atoms separated by distillation from reformed gasoline obtained by a catalytic reforming apparatus. The reaction was conducted under the conditions of a pressure of 3.0 MPa, a temperature of 420° C., an LHSV of 3.0 h⁻¹, a hydrogen/oil ratio of 800 Nm³/m³, and an aromatic hydrocarbons of 9 or more carbon atoms/benzene molar ratio of 4. Table 1 shows the reaction results obtained after 72 and 240 hours from the start of reactions, respectively.

### Comparative Example 1

The catalyst carrier prepared in Example 1 was allowed to carry 3% by mass of Ni (based on the total mass of the catalyst) in terms of Ni metals using an aqueous solution of nickel nitrate by the Incipient Wetness method, dried and calcinated to prepare a catalyst. Then, prior to the following reaction, the resulting catalyst was subjected to a sulfurization treatment at 340° C. for 2 hours with a hydrogen gas containing 1% by volume of hydrogen sulfide based on the volume of the catalyst. A reaction experiment was conducted in the same manner as in Example 1, and the results are shown in Table 1.

### Comparative Example 2

Catalyst preparation, pretreatment, and a reaction experiment were conducted in the same manner as in Example 1, except that hydrogen-ion-type ZSM-5 in which the maximum pore diameter of its micropores was 0.54 nm and the SiO₂/Al₂O₃ ratio was 26 was used in place of the hydrogen-ion-type mordenite (HSZ690HOA, obtained from Toso K.K.) in which the maximum pore diameter of its micropores was 0.70 nm and the SiO₂/Al₂O₃ ratio was 203. Table 1 shows the results.

TABLE 1

| | | Example 1 | | Comp. Ex. 1 | | Comp. Ex.2 | |
|---|---|---|---|---|---|---|---|
| Catalyst | | | | | | | |
| Zeolite | Raw | Mordenite | | Mordenite | | ZSM-5 | |
| silica/alumina molar ratio | Material | 203 | | 203 | | 26 | |
| Na ion exchange | Oil | None | | None | | None | |
| Metal and Amount thereof to be | | Mo, 3.0 | | Ni, 3.0 | | Mo, 3.0 | |
| carried (% by mass) | | | | | | | |
| Running Time (hour) | | 72 | 240 | 72 | 240 | 72 | 240 |

| | | | |
|---|---|---|---|
| Reaction Pressure(Mpa) | 3.0 | 3.0 | 3.0 |
| Reaction Temperature (° C.) | 420 | 420 | 420 |
| LHSV | 3.0 | 3.0 | 3.0 |
| Hydrogen/oil ratio (Nm³/m³) | 800 | 800 | 800 |

| | |
|---|---|
| Distillation Property | |
| (ASTM-D86 method) | |
| IBP/5% | 86/100 |
| 10%/30% | 111/147.5 |
| 50%/70% | 163/166 |
| 90%/95% | 173/180 |
| EP | 232.5 |

| | | | | | | | |
|---|---|---|---|---|---|---|---|
| Composition (% by mass) | | | | | | | |
| Methane | 0.00 | 0.14 | 0.17 | 0.16 | 0.11 | 0.06 | 0.04 |
| Ethane | 0.00 | 7.72 | 7.48 | 8.67 | 6.17 | 3.14 | 2.18 |
| Propane | 0.00 | 5.07 | 5.66 | 5.70 | 4.05 | 2.06 | 1.43 |
| Butane | 0.00 | 1.72 | 1.50 | 1.93 | 1.37 | 0.70 | 0.49 |
| Pentane | 0.00 | 0.34 | 0.25 | 0.38 | 0.27 | 0.14 | 0.10 |
| Hexane | 0.00 | 0.08 | 0.07 | 0.09 | 0.07 | 0.03 | 0.02 |
| Heptane | 0.00 | 0.05 | 0.06 | 0.05 | 0.04 | 0.02 | 0.01 |
| Benzene | 22.46 | 10.63 | 10.79 | 12.17 | 14.65 | 17.61 | 18.91 |
| Toluene | 0.00 | 30.07 | 27.37 | 17.74 | 15.04 | 10.52 | 6.31 |
| C8 Aromatics | 0.02 | 29.60 | 28.68 | 18.95 | 15.11 | 9.19 | 6.08 |
| C9 Aromatics | 64.90 | 12.06 | 14.26 | 23.16 | 31.61 | 44.29 | 51.95 |
| Trimethylbenzene | 34.29 | 9.88 | 10.73 | 13.54 | 16.96 | 26.72 | 29.65 |
| Others | 30.61 | 2.18 | 3.53 | 9.61 | 14.65 | 17.57 | 22.30 |
| C10+ aromatics | 12.62 | 2.51 | 3.70 | 11.00 | 11.51 | 12.24 | 12.46 |
| Naphthalene | 0.69 | 0.08 | 0.10 | 0.57 | 0.60 | 0.68 | 0.69 |
| Total | 100.0 | 100.00 | 100.0 | 100.00 | 100.00 | 100.0 | 100.00 |
| Benzene Conversion Ratio (%) | | 52.7 | 52.0 | 45.8 | 34.8 | 21.6 | 15.8 |
| C9 Aromatics Conversion Ratio | | | | | | | |
| (%) | | 81.4 | 78.0 | 64.3 | 51.3 | 31.8 | 19.9 |
| Trimethylbenzene | | 71.2 | 68.7 | 60.5 | 50.5 | 22.1 | 13.5 |
| Others | | 92.9 | 88.5 | 68.6 | 52.1 | 42.6 | 27.1 |
| C10+ Aromatics Conversion Ratio | | | | | | | |
| (%) | | 80.1 | 70.7 | 12.8 | 8.8 | 3.0 | 1.3 |
| Naphthalene | | 88.1 | 85.6 | 17.6 | 13.2 | 1.8 | 0.6 |
| Variation in Benzene Conversion | | | 98.6 | | 75.9 | | 73.2 |
| Ratio (%) | | | | | | | |
| Variation in C9 Aromatics | | | 95.8 | | 79.7 | | 62.8 |
| Conversion Ratio (%) | | | | | | | |
| Trimethylbenzene | | | 96.5 | | 83.5 | | 61.3 |
| Others | | | 95.3 | | 76.0 | | 63.7 |
| Variation in C10+ Aronatics | | | 88.2 | | 68.8 | | 42.1 |
| Conversion Ratio (%) | | | | | | | |
| Naphthalene | | | 97.1 | | 75.0 | | 35.0 |

Apparent from the results given in Table 1, in Example 1 where the catalyst according to the present invention is used, the conversion ratio of benzene and aromatic hydrocarbons of 9 or more carbon atoms is higher and the proportion of toluene and C8 aromatic hydrocarbons (objective products) is larger after running times of 72 hours as well as 240 hours as compared with Comparative Example 1 (different in the metal used from the present invention, Ni) It is also clearly recognized that the variations in benzene conversion ratio and in C9 aromatics conversion ratio (the ratio of conversion ratio after 240 hours to conversion ratio after 72 hours) is high, indicating less catalytic deterioration. The same is true for trimethylbenzene and other compounds among C9 aromatic hydrocarbons. In addition, the conversion ratios of aromatic hydrocarbons of 10 or more carbon atoms are significantly high. Among them, the conversion ratio of naphthalene is also considerably high. Further, any of the conversion ratio, yield, and stability are significantly superior as compared with Comparative Example 2 (different in the maximum pore diameter from the present invention, ZSM-5).

These results reveal that this catalyst is very excellent in conversion of the material oil containing a component with a boiling point exceeding 210° C.

While the presently preferred embodiments of the present invention have been shown and described, it will be understood that the present invention is not limited thereto, and that various changes and modifications may be made by those skilled in the art without departing from the scope of the invention as set forth in the appended claims.

What is claimed is:

1. A catalyst for converting aromatic hydrocarbons of 9 or more carbon atoms in a material oil containing a component with a boiling point exceeding 210° C. into toluene and aromatic hydrocarbons of 8 carbon atoms in the presence of hydrogen, said catalyst comprising: a carrier containing one or more than one zeolites in which the maximum pore diameter of micropores is in a range of 0.6 to 1.0 nm; and one or more than one metals selected from the Group VIA metals of the Periodic Table or compounds thereof.

2. The catalyst according to claim 1, wherein said metal is Mo.

3. The catalyst according to claim 2, wherein said zeolite is mordenite.

4. The catalyst according to claim 3, a molar ratio of SiO₂/Al₂O₃ in said zeolite is 10 or more.

5. The catalyst according to claim 1, wherein said metal is present in the form of metal oxide.

6. The catalyst according to claim 5, wherein said material oil contains benzene.

7. A method of converting aromatic hydrocarbons of 9 or more carbon atoms in a material oil containing a component with a boiling point exceeding 210° C. into toluene and aromatic hydrocarbons of 8 carbon atoms in the presence of hydrogen using a catalyst, said catalyst comprising: a carrier containing one or more than one zeolites in which the maximum pore diameter of micropores is in a range of 0.6 to 1.0 nm; and one or more than one metals selected from the Group VIA metals of the Periodic Table or compounds thereof.

8. The method according to claim 7, wherein said metal is Mo.

9. The method according to claim 8, wherein said zeolite is mordenite.

10. The method according to claim 9, a molar ratio of SiO₂/Al₂O₃ in said zeolite is 10 or more.

11. The method according to claim 7, wherein said metal is present in the form of metal oxide.

12. The method of converting aromatic hydrocarbons according to claim 7, wherein aromatic hydrocarbons of 9 or more carbon atoms and benzene in the material oil containing a component with a boiling point exceeding 210° C. are converted into toluene and aromatic hydrocarbons of 8 carbon atoms in the presence of hydrogen.

13. The method according to claim 12, wherein said metal is Mo.

14. The method according to claim 13, wherein said zeolite is mordenite.

15. The method according to claim 14, a molar ratio of SiO₂/Al₂O₃ in said zeolite is 10 or more.

16. The method according to claim 12, wherein said metal is present in the form of metal oxide.
